(19) United States
(12) Patent Application Publication
(10) Pub. No.: US 2025/0032647 A1
(43) Pub. Date: Jan. 30, 2025

(54) USE OF MULTIPHOTON EXCITATION FOR FLUORESCENCE IMAGING OF STAINED CALCIFIED MINERALS

(71) Applicant: UNIVERSITY OF MARYLAND, BALTIMORE, Baltimore, MD (US)

(72) Inventors: Richard THOMPSON, Baltimore, MD (US); Huihui ZENG, Ellicott City, MD (US); Krishanu RAY, Ellicott City, MD (US)

(21) Appl. No.: 18/919,564

(22) Filed: Oct. 18, 2024

Related U.S. Application Data
(63) Continuation of application No. PCT/US2023/020166, filed on Apr. 27, 2023.
(60) Provisional application No. 63/335,274, filed on Apr. 27, 2022.

(51) Int. Cl. A61K 49/00

(52) U.S. Cl. CPC A61K 49/0021; A61K 49/0052

(57) ABSTRACT

Deposition of calcium-containing minerals such as hydroxyapatite and whitlockite in the subretinal pigment epithelial (sub-RPE) space is linked to the development of, and progression to, end-stage age-related macular degeneration (AMD). Calcification in the sub-RPE space is also directly linked to other diseases such as Pseudoxanthoma elasticum (PXE). These deposits bind to tetracycline derivatives and can be imaged by fluorescence lifetime contrast using multiphoton (infrared) excitation.

### CROSS REFERENCE TO RELATED APPLICATIONS

This application is a bypass continuation application claiming priority to International Patent Application No. PCT/US2023/020166 filed on 27 Apr. 2023, which claims priority U.S. Provisional Patent Application No. 63/335,274 filed on Apr. 27, 2022 in the name of Richard Thompson et al. and entitled “Use of Multiphoton Excitation for Fluorescence Imaging of Stained Calcified Materials,” both of which are hereby incorporated by reference herein in their entirety.

### STATEMENT OF FEDERALLY SPONSORED RESEARCH

This invention was made with government support under Grant Number EY030443 and AI172487 awarded by the National Institutes of Health. The government has certain rights in the invention.

### FIELD

The present invention relates to predicting, detecting, and diagnosing age-related macular degeneration (AMD), Alzheimer's disease (AD) and other diseases associated with hydroxyapatite-containing deposits in the retina and other tissues of a subject. In particular, the presently disclosed invention provides for imaging using fluorescence lifetime contrast using multiphoton (infrared) excitation to identify the presence of hydroxyapatite (HAP) and/or HAP spherules that may be deposited in the sub-retinal pigment epithelium (RPE) and contribute to the growth of sub-RPE deposits associated with AMD and/or AD.

### BACKGROUND

Age-related macular degeneration (AMD) is the most common cause of legal blindness in developed countries, affecting over ten million people in the United States. There is no cure for AMD and although the 15% of cases comprising the most destructive, “wet” form of AMD can be slowed by treatment with VEGF inhibitors, for 85% of cases most patients present with irreversible vision loss.

Accumulation of protein- and lipid-containing deposits external to the RPE is common in the aging eye, and has long been viewed as the hallmark of age-related macular degeneration (AMD) and/or Alzheimer's disease. In the eye, the Bruch's membrane (BM), which is interposed between the RPE and choroid, becomes thickened with age. This thickening is associated with accumulation of deposits, termed generally as sub-RPE deposits, that may be focal (recognized clinically as sub-retinal deposits or drusen) or diffuse (basal laminar or linear deposits, depending on whether they are present internal or external to the RPE basement membrane). These deposits contain several different proteins derived from sources both in the retina and the serum, notably including beta-amyloid, complement factor H, serum albumin, vitronectin, apolipoprotein E, and crystallins; some of these proteins are known to avidly bind metal ions such as zinc and calcium. The process occurs maximally in the macula, the locus of highest resolution vision, and is integral to the pathogenesis of age-related macular degeneration (AMD) but remains poorly understood.

Due to this correlation between AMD and sub-RPE deposit formation, substantial effort has been devoted to determining the composition and origin of sub-RPE deposits, with a view to developing better methods of diagnosis, prevention, and treatment for AMD [Sarks, 1999]. Although AMD does not necessarily follow the same course in all patients, it is acknowledged that progression of sub-RPE deposit formation is a major factor in a large proportion of cases.

Several therapies are in development for the treatment of AMD, however, to be most effective these treatments should be started before damage has occurred. Thus, an early diagnostic or screening tool is desirable. It would therefore be advantageous to provide a diagnostic method for predicting, detecting, and diagnosing age-related macular degeneration (AMD), Alzheimer's disease (AD) and other diseases associated with hydroxyapatite-containing deposits in the retina tissue of a subject, wherein the diagnostic method maximizes patient comfort and safety while also being able to identify early calcification.

### SUMMARY

In one aspect, the present invention relates to the use of multiphoton excitation of a fluorescent label for early screening and imaging of HAP in tissue in a subject, by imaging any HAP deposits in said tissue stained by said fluorescent label using a fluorescence detector, e.g., fluorescence ophthalmoscopy, capable of fluorescence lifetime imaging. In one embodiment, excitation with very high peak powers in a picosecond/femtosecond laser pulse using photons having approximately twice the wavelength of those otherwise absorbed by the fluorescent label in question, which can result in excitation of the fluorescent label, is used.

In another aspect, a method of detecting and/or diagnosing age-related macular degeneration or Alzheimer's disease in retina tissue of a subject is described, the method comprising:
administering a hydroxyapatite-selective fluorescent dye to the subject in an amount sufficient for binding to any hydroxyapatite (HAP) deposits or spherules present in retina tissue to form a HAP/fluorescent dye complex;
exciting the HAP/fluorescent dye complex with electromagnetic radiation (EMR), wherein the EMR has a wavelength in a range from about 700 nm to about 1 mm; and
monitoring and/or measuring the lifetime of a fluorescent signal of the HAP/fluorescent dye complex with a fluorescence lifetime imaging device, wherein the HAP/fluorescent dye complex exhibits a longer fluorescence lifetime signal than that of background fluorescence of the retina tissue.

In still another aspect, a method for identifying or labeling of HAP deposits in tissue in a subject is described, the method comprising:
administering a hydroxyapatite-selective fluorescent dye to the subject in an amount sufficient for binding to any hydroxyapatite (HAP) deposits or spherules present in tissue to form a HAP/fluorescent dye complex;
exciting the HAP/fluorescent dye complex with electromagnetic radiation (EMR), wherein the EMR has a wavelength in a range from about 700 nm to about 1 mm; and
monitoring and/or measuring the lifetime of a fluorescent signal of the HAP/fluorescent dye complex with a fluorescence lifetime imaging device, wherein the HAP/fluorescent dye complex exhibits a longer fluorescence lifetime signal than that of background fluorescence of the tissue.

Further features and advantages of the present invention will become evident to those of ordinary skill in the art after a study of the present application.

### BRIEF DESCRIPTION OF THE DRAWINGS

FIG. 1A. Two-photon-excited fluorescence intensity image fit to a single component. Experiments were carried out on a choroid flat-mount, following the removal of the RPE and neurosensory retina (97-year-old donor; cause of death: cardiovascular disease). Sample was labelled with Cl-Tet. Approximate size of the field: 250×250 μm².

FIG. 1B. Two-photon-excited fluorescence lifetime image fit to a single component. Experiments were carried out on a choroid flat-mount, following the removal of the RPE and neurosensory retina (97-year-old donor; cause of death: cardiovascular disease). Sample was labelled with Cl-Tet. Approximate size of the field: 250×250 μm².

FIG. 2A. Fluorescence intensity micrograph of doxycycline-stained drusen in the retina of a 79 year old white male donor.

FIG. 2B. Decay of the indicated region of interest in FIG. 2A.

FIG. 3A. Close-up of the druse in FIG. 2A (fluorescence intensity).

FIG. 3B. Best two component fits to the pink region of interest at position X=−33 μm, Y=−17 μm in FIG. 3A with both lifetimes floating.

FIG. 4A. Close-up of the druse in FIG. 2A (single component fluorescence lifetime).

FIG. 4B. Best two component fits to the pink region of interest at X=−33 μm, Y=−17 μm in FIG. 3A, with one lifetime fixed to 3.7 nsec.

FIG. 5A. Close-up of synthetic HAP stained with Cl-tetracycline. 780 nm excitation pulses of 90 fsec duration at 50 MHz repetition rates.

FIG. 5B. Best two component fit to the decay in the highlighted region of interest, yielding a mean lifetime of 1.31 nsec, χ2=1.4.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Subject” and “patient” as used herein interchangeably refers to any vertebrate, including, but not limited to, a mammal (e.g., a bear, cow, cattle, pig, camel, llama, horse, goat, rabbit, sheep, hamster, guinea pig, cat, tiger, lion, cheetah, jaguar, bobcat, mountain lion, dog, wolf, coyote, rat, mouse, and a non-human primate (for example, a monkey, such as a cynomolgus or rhesus monkey, chimpanzee, etc.) and a human). In some embodiments, the subject is a human. It should be appreciated herein that a “fluorescent label” comprises a fluorophore.

The terms “diagnosing” and “diagnosis,” as used herein, refer to methods by which the skilled artisan can estimate and even determine whether or not a subject is suffering from a given disease or condition. Along with diagnosis, clinical “prognosis” or “prognosticating” is also an area of great concern and interest. It is important to know the relative risk associated with particular conditions in order to plan the most effective therapy. If an accurate prognosis can be made, appropriate therapy, and in some instances less severe therapy or more effective therapy, for the patient can be chosen to benefit the patient.

As defined herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like.

“Fluorescence lifetime” as used herein broadly refers to the emissive lifetime of the photoluminescent species, being the average time the photoluminescent species spends in the excited state between excitation and emission of the photoluminescence, and thus the inverse of the emissive rate.

As defined herein, a “fluorescent dye” includes, but is not limited to, tetracycline derivatives and fluorescent calcification stains (e.g., Bone-Tag, OsteoSense, Xylenol Orange, 2,7-dichlorofluorescein methyl ester, 5,10,15,20-tetrakis [m-phenylphosphonic acid] porphyrin (m-H8TPPA), and Alizarin Red S). As used herein, the term “tetracycline derivative” includes tetracycline as well as its derivatives, and any combination thereof, including, but are not limited to, chlortetracycline, demeclocycline, doxycycline, methacycline, oxytetracycline, anhydrochlortetracycline, anhydrotetracycline, cetocycline and chelocardin. Oxytetracycline and doxycycline are approved for intravenous application in humans by the Federal Drug Administration (FDA). Demeclocycline, methacycline, tetracycline, oxytetracycline and doxycycline are approved for oral delivery. Chlortetracycline is approved for local topical administration but was previously administered orally. The tetracycline derivatives may be delivered orally, topically, intravenously, intravitreally, or intraocularly to detect retinal HAP deposits as described herein.

As used herein, the “HAP/fluorescent dye complex” corresponds to a complex that may be associated via noncovalent or covalent bonds. It should be appreciated by the person skilled in the art that the fluorescent dye may exhibit a change in its fluorescence excitation or emission wavelength (color), quantum yield, anisotropy (i.e., polarization) and/or lifetime upon binding to the mineral.

The present inventors have previously demonstrated that sub-RPE deposits (the best known of which are drusen) contain microscopic deposits of calcium minerals, particularly hydroxyapatite (HAP), which is a highly insoluble basic form of calcium phosphate (3Ca₃(PO₄)₂Ca(OH)₂) and the principal mineral in bone and teeth, and whitlockite (WHT). HAP is marked by the additional hydroxide ions in its structure, is generally formed under more basic conditions than the mono- and dibasic calcium phosphates seen in tissue calcification and is much less soluble and more stable than the other calcium phosphate forms, particularly under physiological conditions, e.g., pH. In some embodiments, the HAP deposits are crystalline. In some embodiments, the HAP deposits are microcrystalline. In some embodiments, the HAP deposits are amorphous. It should be appreciated by the person skilled in the crystal structure arts that imperfections in the HAP crystal structure, e.g., because of an atom substitution or deletion, is common, but the crystal is still considered HAP.

Although used throughout this application, the term “hydroxyapatite” or “HAP” is intended to encapsulate HAP and other known calcium phosphate minerals including, but not limited to, whitlockite (Ca₉(MgFe)(PO₄)₆(PO₃OH), octacalcium phosphate (OCP) (Ca₈H₂(PO₄)₆·5H₂O), and dicalcium phosphate dihydrate (CaHPO₄·2H₂O). In other words, in some embodiments, reference to HAP corresponds to HAP per se. In some embodiments, reference to HAP corresponds to at least one of whitlockite, OCP, and DCPD. In some embodiments, reference to HAP corresponds to HAP and at least one of whitlockite, OCP, and DCPD.

As drusen are a clinical hallmark of AMD, the most common cause of irreversible vision loss in the elderly in developed countries, their early detection could be paramount for developing new intervention strategies. Calcification is common in all forms of sub-RPE deposits, and this calcification frequently takes the form of spherules of diameter circa one micron that were often coated with proteins characteristic of drusen, and these mineral deposits may nucleate the growth of drusen [Thompson, 2015]. Larger calcified deposits (termed nodules) are an important risk factor for developing advanced AMD (either geographic atrophy or choroidal neovascularization) within one year (odds ratio 6.4:1) [Tan, 2018]. Advantageously, these mineral deposits can be imaged in situ. The large HAP nodules (tens of microns) provide good contrast for multimodal imaging; however, even with adaptive optics imaging, the micron-sized spherules are too small to resolve. The most commonly used visualization methods for tissue calcification generally are by radiography or ultrasound, but at present their resolution is also well below what is needed to detect early calcification.

Some tetracyclines are known to stain HAP with an accompanying fluorescence intensity increase. However, their emission wavelength overlaps with the well-known tissue autofluorescence in the eye [Zinkernagel, 2019], compromising sensitivity and specificity, especially for the micron-sized calcification. There are selective fluorescent stains for HAP which offer improved sensitivity by emitting at near infrared (NIR) wavelengths (above about 810 nm), where retinal tissue autofluorescence is usually minimal. Their excitation at NIR wavelengths would also improve tissue penetration, patient comfort and safety. However, these stains were less attractive for in vivo studies since their pharmacokinetics, toxicity, and ADME (absorption, distribution, metabolism, and excretion) properties are largely unknown, particularly in primates. Moreover, the heptamethine cyanine fluorescent moieties of these infrared dyes are noted as photosensitizers in other circumstances [Hamer, 1964], potentially compromising safety. Finally, to our knowledge the cyanine stains could only be administered by injection. By comparison, tetracycline antibiotics are widely considered safe, and have well-known pharmacokinetics, toxicity, and ADME profiles; moreover, nearly all are orally bioavailable.

The present inventors have also previously shown that binding of certain tetracyclines to HAP was accompanied by a significant increase in apparent quantum yield and fluorescence lifetime [Szmacinski, 2020]. Therefore, it is possible to distinguish drusen from the background fluorescence of post-mortem retinal tissues (minus the neural retina) due to the large difference in the lifetime of chlortetracycline (Cl-Tet, Aurcomycin, CAS no. [57-62-5]), bound to HAP (1.6 nsec) compared with the known retinal autofluorescence lifetime (0.4 nsec; reviewed in [Zinkernagel, 2019]) using fluorescence lifetime imaging microscopy (FLIM, [Szmacinski, 1994]). The pioneering development of fluorescence lifetime imaging ophthalmoscopy (FLIO) by Schweitzer, Zinkernagel, and their colleagues and its demonstration in living humans (reviewed in [Zinkernagel, 2019]) suggests that HAP could be imaged in situ following oral administration of a tetracycline, and predict the development of drusen deposition and growth, as an early marker for age-related macular degeneration.

By comparison with chlortetracycline, doxycycline (Doxy, CAS [17086-28-1]) offers even better resolution of its emission when bound to HAP (3.8 nsec compared to the 0.4 nsec of tissue background) with a commensurate increase in quantum yield. However, there is a significant drawback: the maximum of its excitation is at approximately 390 nm. This is problematic both because of the overt safety issue of illuminating the eye with ultraviolet light, but also because the shorter wavelength excitation typically adds to the autofluorescence background, especially in the aqueous and vitreous humors, lens, and cornea.

Multiphoton absorption was predicted theoretically by Göppert-Mayer [Göppert-Mayer, 1931] and applied to fluorescence microscopy by Webb and colleagues [Denk, 1990]. Under conditions of very high peak power (typically realized in ultrashort mode-locked laser pulses in the pico- to femtosecond ranges), a molecule can simultaneously absorb two (or more) longer wavelength photons as if they were a single photon of twice the energy and half the wavelength, and thus be raised to an excited state. If fluorescent, the molecule will ordinarily emit as if it had absorbed a single photon of higher energy in the usual way. It is known to the art that the wavelength-dependence of the propensity of fluorophores to undergo such two- or three-photon excitation is not necessarily equal or even proportional to the molecule's one-photon absorption extinction coefficient (ε), but rather to the molecule's multiphoton excitation cross section, expressed in units of Göppert-Mayers (GM). An organic fluorophore such as pyrene (being centrosymmetric) may have effectively zero two-photon excitation cross section, whereas a bright fluorophore such as Rhodamine B has a 200 GM cross section, and certain squaraine-rotaxanes have cross-sections as large as 10,000 GM.

Towards that end, the present invention provides for methods for labeling and detecting HAP deposits in tissue including, but not limited to, retina tissue. Embodied methods include contacting a tissue sample of a subject with a fluorescent dye (e.g., a tetracycline derivative), irradiating the tissue sample with electromagnetic radiation having a wavelength preferably at least about 700 nm, and detecting a signal from the fluorescent dye after binding with HAP deposits (i.e., after a HAP/fluorescent dye complex, or a HAP/tetracycline derivative complex is formed). In some embodiments, multiphoton excitation is used to irradiate the HAP/fluorescent dye complex. In some embodiments, two-photon excitation is used to irradiate the HAP/fluorescent dye complex. In some embodiments, a HAP-selective fluorescent dye/label is used in combination with a fluorescence lifetime imaging device for early detection of HAP spherules or HAP deposits, e.g., in the retina tissue of a subject, including the peripheral and/or macula tissue, wherein the HAP spherules or HAP deposits initiate or support the growth of sub-RPE deposits and correlate with age-related macular degeneration and/or Alzheimer's disease. The presence of, or change in the fluorescence properties of, to include characteristic wavelength(s) of excitation or emission, anisotropy (polarization), and/or lifetime, the signal from the fluorescent dye indicates the presence of HAP deposits, such as drusen, sub-retinal deposits or combinations thereof. Advantageously, the methods described herein permit earlier and accurate detection of such deposits and earlier detection of macular degeneration, such as AMD, and identification of individuals with an increased likelihood of developing macular degeneration such as AMD. Moreover, the methods described herein can be used to monitor the progression or regression of sub-retinal deposits by, for example, fluorescence ophthalmoscopy, using multiphoton (infrared) excitation.

Towards that end, in a first aspect, the present invention broadly relates to the use of multiphoton excitation of a fluorescent label for early screening and imaging of HAP in tissue in a subject, by imaging any HAP deposits in said tissue stained by said fluorescent label using a fluorescence detector, e.g., fluorescence ophthalmoscopy, capable of fluorescence lifetime imaging. In one embodiment, excitation with very high peak powers in a picosecond/femtosecond laser pulse using photons having approximately twice the wavelength of those otherwise absorbed by the fluorescent label in question, which can result in excitation of the fluorescent label, is used.

In one embodiment of the first aspect, the present invention relates to the use of multiphoton excitation of a fluorescent label for early screening and imaging of HAP in retina tissue, that being the peripheral retina or the macula, to predict or diagnose age-related macular degeneration and/or Alzheimer's disease, by imaging any HAP deposits in the retina stained by said fluorescent label using a fluorescence detector, e.g., fluorescence ophthalmoscopy, capable of fluorescence lifetime imaging. In one embodiment, excitation with very high peak powers in a picosecond/femtosecond laser pulse using photons having approximately twice the wavelength of those otherwise absorbed by the fluorescent label in question, which can result in excitation of the fluorescent label, is used.

In another embodiment of the first aspect, the present invention relates to the imaging of other calcification in the body of the subject including, but not limited to, calcifications in breast cancer, atherosclerosis, and the cardiovascular system.

Notably, the fluorescent labels chosen substantially bind to HAP and have a GM cross section that is capable of multiphoton excitation. In some embodiments, tetracycline derivatives are used as a fluorescent label that binds substantially and specifically to HAP and which allows its presence to be identified by means of a fluorescent signal. Advantageously, tetracycline derivatives exhibit a change in the signal upon binding to the HAP, that being, the tetracycline derivative exhibits a substantial increase in fluorescence lifetime upon binding to HAP in tissue, thus permitting HAP to be identified by fluorescence lifetime imaging methods known in the art.

In some embodiments, the HAP-selective fluorescent dye comprises chlortetracycline and the presence of a fluorescence lifetime signal of from about 1.4 to about 1.9 nsec indicates the presence of HAP deposits. In some embodiments, the HAP-selective fluorescent dye comprises doxycycline and the presence of a fluorescence lifetime signal of from about 2.0 to about 4.2 nsec indicates the presence of HAP deposits.

In some embodiments, the tetracycline derivative, as a fluorescent label, may be formulated as a pharmaceutical composition for delivery to the eye, for example for topical delivery, as an eye drop comprising an eye drop solution, or for intravitreal or other intraocular injection, or administered orally. In some embodiments, the tetracycline derivative may be formulated as a pharmaceutical composition for systemic administration, for example as an intravenous injection or the like. Systemic administration may also be appropriate when the target tissue is in the eye. The pharmaceutical composition comprising the fluorescent label may be combined with any appropriate pharmaceutically acceptable carrier, adjuvants, and/or excipients necessary or advantageous for the delivery method selected.

In some embodiments, the pharmaceutical composition comprising the HAP-selective fluorescent dye is administered to the subject, and the imaging with the fluorescence lifetime imaging device occurs about 1 hr to about 5 days subsequent to said administration, or about 1 hr to about 4 days subsequent to said administration, or about 1 hr to about 72 hours subsequent to said administration, or about 1 hr to about 48 hours subsequent to said administration, or about 1 hr to about 24 hours subsequent to said administration, or about 24 hr to about 48 hr subsequent to said administration, or about 24 hr to about 72 hr subsequent to said administration, or about 24 hr to about 4 days subsequent to said administration, or about 48 hr to about 72 hr subsequent to said administration, or about 72 hr to about 4 days subsequent to said administration, or about 4 days to about 5 days subsequent to said administration, or about 12 hr to about 36 hr subsequent to said administration, as readily determined by the person skilled in the art.

Suitable pharmaceutical compositions include aqueous and non-aqueous sterile injection solutions that comprise tetracycline derivatives and further comprise at least one of antioxidants, buffers, bacteriostats, bactericidal antibiotics and solutes, that render the formulation isotonic with the bodily fluids of the patient; or aqueous and non-aqueous sterile suspensions, which can further include at least one of suspending agents and thickening agents. The pharmaceutical compositions can take such forms as suspensions, solutions, syrups or emulsions in oily or aqueous vehicles, and can contain formulatory agents such as suspending agents (e.g., sorbitol syrup, cellulose derivatives or hydrogenated edible fats), emulsifying agents (e.g. lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), stabilizing and/or dispersing agents. Alternatively, the fluorescent label can be in powder form for constitution with a suitable vehicle, e.g., sterile pyrogen-free water, before use.

Pharmaceutical compositions for oral administration can be obtained by combining the tetracycline derivative with suitable carriers. Suitable carriers include at least one of fillers (such as sugars, for example, lactose, saccharose, mannitol or sorbitol), cellulose preparations, calcium phosphates (e.g., tricalcium phosphate or calcium hydrogen phosphate), binders (such as starch pastes, using, for example, corn, wheat, rice or potato starch, gelatin, tragacanth, methylcellulose and/or polyvinylpyrrolidone) and disintegrators (such as the above-mentioned starches, also carboxymethyl starch, cross-linked polyvinylpyrrolidone, agar or alginic acid or a salt thereof, such as sodium alginate). Coloring substances or pigments may also be added, for example for the purpose of identification or to indicate different doses of the pharmaceutical composition comprising the tetracycline derivative. Other orally administrable pharmaceutical compositions include dry-filled capsules made of gelatin, and also soft, sealed capsules made of gelatin and a plasticizer, such as glycerol or sorbitol. Preparations for oral administration can be suitably formulated to give controlled release of the active compound. For buccal administration the pharmaceutical compositions can take the form of tablets or lozenges formulated in conventional manner.

The pharmaceutical compositions can be presented in unit-dose or multi-dose containers, for example sealed ampoules and vials, and can be stored in a frozen or freeze-dried (lyophilized) condition requiring only the addition of sterile liquid carrier immediately prior to use. In some embodiments the pharmaceutical compositions are contained in containers that can dispense the solution in a drop wise manner. Such containers are particularly beneficial for administering eye drop formulations comprising the present labels and compositions.

Monitoring and/or measuring the lifetime of a fluorescent signal of the HAP/fluorescent label complex can be conducted by fluorescence lifetime imaging devices employing time and frequency domain technologies, time-correlated single photon counting, time-gated photon counting with laser scanning, streak cameras, point scanning and wide-field imaging techniques, such as a modulated charge-coupled device. Advantageously, using fluorescence lifetime imaging to measure the lifetime fluorescence of binding to HAP and overcomes the fluorescent interference (“background fluorescence”) from pigment granules and other molecules including lipofuscin in the RPE layer of the retina and other portions of the retina. As such, the emission from the fluorescent label with a suitably longer lifetime could be resolved from shorter lifetime background fluorophores including, in some cases, the fluorescent label unbound or nonspecifically bound. Fluorescence lifetime imaging creates fluorescence images wherein the contrast of the image arises not from differences in emission intensity, but rather differences in fluorescence lifetime. Imaging devices are available with this capability, for example, from ISS, Inc., Urbana, IL; Nikon, Inc., Garden City, NY; Becker und Hickl, GmbH, Berlin, Germany; Heidelberg Engineering GmbH, Heidelberg, Germany; and PicoQuant GmbH, Berlin, Germany.

Fluorescence-lifetime imaging is an imaging technique for producing an image based on the differences in the exponential decay rate of the fluorescence from a fluorescent sample, and is able to distinguish between the different fluorophores in a biological sample. Due to the broad and overlapping emission spectra of many fluorophores or endogenous fluorophores, it is often difficult to quantitatively measure the concentrations of these different species contributing to the fluorescence emission signal by spectral filtering alone. Fluorescence lifetime imaging is based on the fact that every fluorophore has a characteristic excited-state lifetime, t, or time for the molecule to decay from the excited electronic state to the ground state. Thus, for fluorescence lifetime imaging, one is collecting an image where the basis of contrast in the image comprises differences in the fluorescence lifetime(s) in the picture elements (pixels) making up the image, and not necessarily the fluorescence intensity or color.

Lifetime information can be measured either by time-domain or frequency-domain methods, both of which are well known to the art for decades. In the time-domain technique, a pulsed excitation source is used to excite the fluorophore of interest in the biological sample. The subsequent time profile of the fluorescence emission is typically measured using time gating or time-correlated single photon-counting techniques, with the lifetime t in the ideal case of a single component determined from the time-dependent emission I(t) by the expression I(t)=I₀e^{-t/τ}. When multiple components with different lifetimes are present, the expression is more complex and the time-dependent emission data are typically fit to an assumed decay law to determine the lifetimes and fractional intensities (or preexponential factors) of the components. In frequency-domain, a constant frequency, amplitude-modulated excitation source is often employed. The lifetime of the fluorophore causes the emitted fluorescence signal to be modulated at the same frequency but with a phase delay and lower modulation relative to the excitation light. Measurement of this phase delay using phase-sensitive detection (such as a lockin amplifier) or other means will then give the value of the lifetime of a single component, τ, by the relation tan φ=ωτ, where φ is the phase offset and ω is the modulation frequency. Similarly, the modulation m of the emission of a single component with respect to that of the excitation (m=mₑₘᵢₛₛ/m_{exc}) is also a simple function of the lifetime and modulation frequency: m=(1+ω²+τ²)^{-1/2}. When multiple components with differing lifetimes are present these simple relations no longer hold, and phase delays and modulations measured at several different modulation frequencies are measured and also commonly fit to assumed decay laws to determine the lifetimes and fractions of the components. Therefore, collecting the fluorescence lifetime image typically entails (in the time domain) collecting the time-dependent intensity for individual pixels or (in the frequency domain) phase and modulation data at multiple frequencies for individual pixels, then processing and displaying the lifetimes or lifetime-derived information as an image. In the case of multiple lifetime components being present, such lifetime-derived information may be an average lifetime computed by fitting the time decay of the pixel and obtaining the resulting lifetimes and fractions. In the frequency domain the average lifetime may also be computed but it is often convenient to highlight a subset of pixels in the image having a particular range of phase and modulation at some suitable frequency as indicating the lifetimes and fractions of the emitters present in those pixels. This is fast and convenient since in this case no complex fitting process need be implemented pixel by pixel, as it must be in the time domain.

In some embodiments, recovering the actual values of the lifetimes of individual pixels may not be important: rather, identifying pixels in the image where the fluorescent label is adhering to calcification in the drusen, which is essentially a bimodal, black/white mapping, may be used. For example, in some embodiments, use of the phasor plot introduced by Redford and Clegg [Redford, 2005], that enables pixels exhibiting particular lifetime properties to be highlighted, is well suited. A commercially available software program plots different points on the phasor plot using the phases and modulations at a particular frequency of individual pixels in an image, and one can highlight areas in the image based on their lifetime properties by selecting a subset of points with a small circle to indicate the region of interest (in Δφ and m space). Importantly, data in the time domain may also be transformed to construct a phasor plot. In some embodiments, for lifetime data acquired in the frequency domain, the use of classic phase-sensitive detection at a suitable modulation frequency [Lakowicz, 1983] can also be used in an imaging format to discriminate between pixels on the basis of their lifetime properties.

In some embodiments, the fluorescence lifetime imaging device comprises at least one excitation source that generates radiation and at least one detector that receives the detected radiation emitted from the sample. The excitation source preferably contains a laser that emits pulsed excitation radiation and an adjusting apparatus for adjusting the pulse repetition rate to the specific lifetime properties of the sample. In some embodiments, pulsed excitation sources that exhibit pulse durations in the picosecond to femtosecond range are used to excite the HAP/fluorescent dye complex. For frequency and time domain measurements other light sources are useful including, but not limited to, amplitude-modulated (either internally or externally) lasers, light-emitting diodes, ion lasers, lamps, and mode-locked lasers. Similarly, numerous detectors are known to the art for frequency and time domain fluorescence measurements, including diodes, diode arrays, photomultiplier tubes, microchannel plate photomultipliers, avalanche photodiodes, and the like.

In some embodiments, an effective device uses fluorescence lifetime imaging microscopy (FLIM) or fluorescence lifetime imaging ophthalmoscopy (FLIO), the former of which is commercially available from several sources.

In some embodiments, excitation source wavelengths are about 700 nm to about 1 mm, preferably about 700 nm to about 2500 nm, even more preferably greater than about 700 nm to about 1000 nm. It should be appreciated that wavelengths shorter than 700 nm are useable, but they will be visible to the patient and have high peak power. Pulsed excitation sources (lasers of all types, LEDs, or spark gaps) preferably exhibit pulse durations in the picosecond to femtosecond range, whereas amplitude modulated sources exhibit modulation frequencies in the range 100-fold larger or smaller than the expected emissive rate. In one embodiment, a scanning laser ophthalmoscope comprising a short-pulse laser for multiphoton excitation can be used. In some embodiments, a scanning laser ophthalmoscope comprising a short-pulse laser for multiphoton excitation and optics to correct for the effects of wavelength dispersion on the pulse duration can be used.

In one embodiment, two-photon excitation (TPE) is used. In one embodiment, the HAP/fluorescent label complex is excited with near-infrared electromagnetic radiation (EMR) (about 700 nm to about 2500 nm). Time domain data may be collected by, for example, streak cameras, boxcar integrator, and Time-Correlated Single-Photon Counting (TCSPC). In the frequency domain, the HAP/fluorescent label complex is excited by an amplitude-modulated source of electromagnetic energy. The amplitude-modulated excitation may be modulated in a sinusoidal waveform, or a series of pulses, and the modulation frequency is typically within one-hundred fold of the emissive rate. The emission waveform is demodulated and phase-shifted with respect to the excitation waveform. For a single emitting photoluminescent species, the lifetime is a simple function of the frequency and measured phase shift or demodulation. Imaging devices including microscopes and ophthalmoscopes that produce images whose contrast is derived from differences in fluorescence lifetime in the specimen being imaged have been described using both time- and frequency-domain approaches (or both) and some are commercially available. Some employ scanning excitation with point detectors and TCSPC or time-gated detection, others in the frequency domain employ point detectors with scanning excitation, or a camera with modulated gain.

Accordingly, in another embodiment of the first aspect, a method of detecting and/or diagnosing age-related macular degeneration or Alzheimer's disease in retina tissue of a subject is described, the method comprising:
administering a hydroxyapatite-selective fluorescent dye to the subject in an amount sufficient for binding to any hydroxyapatite (HAP) deposits or spherules present in retina tissue to form a HAP/fluorescent dye complex;
exciting the HAP/fluorescent dye complex with electromagnetic radiation (EMR), wherein the EMR has a wavelength in a range from about 700 nm to about 1 mm; and
monitoring and/or measuring the lifetime of a fluorescent signal of the HAP/fluorescent dye complex with a fluorescence lifetime imaging device, wherein the HAP/fluorescent dye complex exhibits a longer fluorescence lifetime signal than that of background fluorescence of the retina tissue.
   In some embodiments, the EMR wavelength is in a range from about 700 nm to about 2500 nm. In some embodiments, multiphoton excitation is used to irradiate the HAP/fluorescent dye complex. In some embodiments, two-photon excitation (TPE) is used to irradiate the HAP/fluorescent dye complex. In some embodiments, the HAP-selective fluorescent dye comprises a species selected from the group consisting of tetracycline, chlortetracycline, demeclocycline, doxycycline, methacycline, oxytetracycline, anhydrochlortetracycline, anhydrotetracycline, cetocycline and chelocardin, preferably chlortetracycline or doxycycline. In some embodiments, the administration of the HAP-selective fluorescent dye is selected from the group consisting of topically, orally, intravenously, intravitreally, and intraocularly. In some embodiments, the HAP-selective fluorescent dye comprises chlortetracycline and the presence of a fluorescence lifetime signal of from about 1.3 to about 1.9 nsec indicates the presence of HAP deposits. In some embodiments, the HAP-selective fluorescent dye comprises doxycycline and the presence of a fluorescence lifetime signal of from about 2.0 to about 4.2 nsec indicates the presence of HAP deposits. In some embodiments, the fluorescence lifetime imaging device uses fluorescence lifetime imaging microscopy (FLIM) or fluorescence lifetime imaging ophthalmoscopy (FLIO). In some embodiments, pulsed excitation sources that exhibit pulse durations in the picosecond to femtosecond range are used to excite the HAP/fluorescent dye complex. In some embodiments, a pharmaceutical composition comprising the HAP-selective fluorescent dye is administered, and the imaging with the fluorescence lifetime imaging device occurs about 1 hr to about 5 days subsequent to said administration. In some embodiments, the lifetime of the fluorescent signal is measured by time-domain or by frequency-domain methods. In some embodiments, the method further comprises scanning the retina tissue of the subject with a fluorescence lifetime imaging device. In some embodiments, the method further comprises obtaining a profile of HAP deposits in the subject's retina tissue, wherein the HAP deposits bound to the fluorescent dye exhibit a longer lifetime compared to background tissue. In some embodiments, the method further comprises using the obtained profile to diagnose or predict age-related macular degeneration and/or Alzheimer's disease in the subject. In some embodiments, the method is used in diagnosing or predicting the likelihood of having or developing age-related macular degeneration and/or Alzheimer's disease in the subject.

In another embodiment of the first aspect, a method for identifying or labeling of HAP deposits in tissue in a subject is described, the method comprising:
administering a hydroxyapatite-selective fluorescent dye to the subject in an amount sufficient for binding to any hydroxyapatite (HAP) deposits or spherules present in tissue to form a HAP/fluorescent dye complex;
exciting the HAP/fluorescent dye complex with electromagnetic radiation (EMR), wherein the EMR has a wavelength in a range from about 700 nm to about 1 mm; and
monitoring and/or measuring the lifetime of a fluorescent signal of the HAP/fluorescent dye complex with a fluorescence lifetime imaging device, wherein the HAP/fluorescent dye complex exhibits a longer fluorescence lifetime signal than that of background fluorescence of the tissue.

In some embodiments, the tissue is retina tissue. In some embodiments, the EMR wavelength is in a range from about 700 nm to about 2500 nm. In some embodiments, multiphoton excitation is used to irradiate the HAP/fluorescent dye complex. In some embodiments, two-photon excitation (TPE) is used to irradiate the HAP/fluorescent dye complex. In some embodiments, the HAP-selective fluorescent dye comprises a species selected from the group consisting of tetracycline chlortetracycline, demeclocycline, doxycycline, methacycline, oxytetracycline, anhydrochlortetracycline, anhydrotetracycline, cetocycline and chelocardin, preferably chlortetracycline or doxycycline. In some embodiments, the administration of the HAP-selective fluorescent dye is selected from the group consisting of topically, orally, intravenously, intravitreally, and intraocularly. In some embodiments, the HAP-selective fluorescent dye comprises chlortetracycline and the presence of a fluorescence lifetime signal of from about 1.3 to about 1.9 nsec indicates the presence of HAP deposits. In some embodiments, the HAP-selective fluorescent dye comprises doxycycline and the presence of a fluorescence lifetime signal of from about 2.0 to about 4.2 nsec indicates the presence of HAP deposits. In some embodiments, the fluorescence lifetime imaging device uses fluorescence lifetime imaging microscopy (FLIM) or fluorescence lifetime imaging ophthalmoscopy (FLIO). In some embodiments, pulsed excitation sources that exhibit pulse durations in the picosecond to femtosecond range are used to excite the HAP/fluorescent dye complex. In some embodiments, a pharmaceutical composition comprising the HAP-selective fluorescent dye is administered, and the imaging with the fluorescence lifetime imaging device occurs about 1 hr to about 5 days subsequent to said administration. In some embodiments, the lifetime of the fluorescent signal is measured by time-domain or by frequency-domain methods. In some embodiments, the method further comprises scanning the tissue of the subject with a fluorescence lifetime imaging device. In some embodiments, the method further comprises obtaining a profile of HAP deposits in the subject's tissue, wherein the HAP deposits bound to the fluorescent dye exhibit a longer lifetime compared to background tissue. In some embodiments, the method further comprises using the obtained profile to diagnose or predict age-related macular degeneration and/or Alzheimer's disease in the subject. In some embodiments, the method is used in diagnosing or predicting the likelihood of having or developing age-related macular degeneration and/or Alzheimer's disease in the subject.

The advantages of TPE are numerous. First, using infrared light pulses can be used to excite the fluorescent label rather than violet-blue or other visible wavelengths, the former being much safer and more comfortable than the latter for the patient. In fact, the two photon excitation wavelengths preferred for the tetracyclines are nearly identical with those used for the commonly used ophthalmoscopic technique, optical coherence tomography (OCT). TPE at these wavelengths has been demonstrated safe for humans in in vivo ophthalmoscopic studies [Palczewska, 2020; Fecks, 2017]. Second, because the peak power is only high enough at the focus of the excitation to produce the two-photon effect, the method is effectively confocal in that there is no excitation of interfering fluorescence elsewhere along the optical axis. Third, the picosecond/femtosecond pulses greater than about 700 nm are invisible (as it is for optical coherence tomography (OCT) and NIR reflectance), and this reduces both risk and patient discomfort and are the ideal excitation for fluorescence lifetime-based imaging, such as fluorescence lifetime ophthalmoscopy (FLIO).

Moreover, it is known that the background fluorescence of scanned retina tissue has a fluorescence lifetime from about 0.4 to about 0.7 nsec and the chlortetracycline derivative bound to HAP deposits has a fluorescence lifetime from about 1.4 to 1.9 nsec, while doxycycline's range is 2.0 to 4.2 nsec. Such comparison provides for identifying the amount and progression of HAP deposits forming in the retina tissue and such method provides for a method that does not include background fluorescence of scanned retina tissue.

In another aspect, the present invention includes a step of generating an image based on the scanned retina and the observed fluorescent signal of any bound tetracycline derivative to HAP deposits. Such an image can be compared with a set of controls, wherein controls include a series of profiles of different levels of HAP deposits representing a particular stage of age-related macular degeneration, including one that does not suffer from age-related macular degeneration.

The present invention also provides for kits comprising a pharmaceutical composition comprising a tetracycline derivative, as described herein.

### Example 1

A feasibility study was performed testing ex vivo staining of cadaver retinas with Chlortetracycline and Doxycycline, either by staining flatmounted retinas or staining intact retinas in cadavers by infusion as previously described [Hegde, et al., in press] and imaging them by FLIM with 2 photon excitation using near-infrared radiation excitation.

For this study eyes from six anonymous deceased donors (3 female, 3 male, mean age 83.7 years) were obtained with informed consent from the Maryland State Anatomy Board under the supervision of the Institutional Review Board. Cadavers were infused through the carotid artery 6-16 hours post mortem with 500 mg/liter chlortetracycline or 250 mg/liter doxycycline in PBS for one hour, followed by a one hour rinse; the eyes were enucleated, the neural retina and RPE removed, and the retinas flatmounted for FLIM, all essentially as previously described [Hegde, et al., in press]. The specimens were imaged with an ISS Q2 (Urbana, IL) fluorescence lifetime imaging microscope through 20×0.7 NA or 60×1.2 NA objectives with 780 nm excitation pulses of 90 fsec duration at 50 MHz repetition rates from a modelocked fiber optic laser with average power up to 12 mW. Data were analyzed and depicted using VistaVision software by ISS.

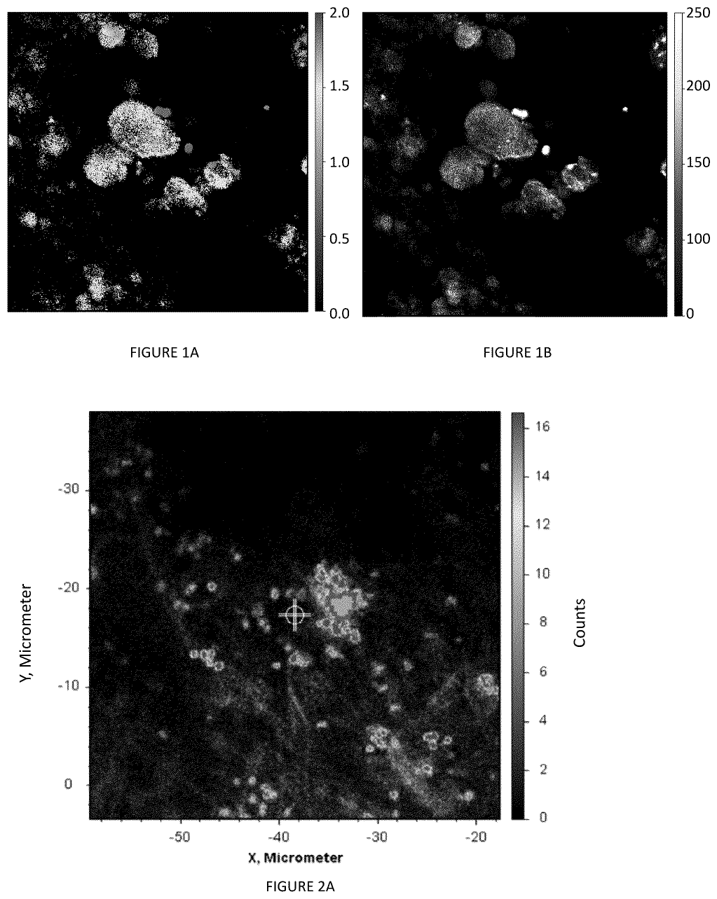

FIGS. 1A and 1B depict a representative flatmounted retina from a 97-year-old Black female donor with the neural retina and RPE removed, exposing the Bruch's membrane-choroid complex stained with Cl-Tet. There are several drusen in the field of view and they stand out in both the intensity (FIG. 1A) and lifetime (FIG. 1B) images. Whereas the intensities of the drusen in FIG. 1A are two- to three-fold larger than the background, the lifetimes of the drusen on FIG. 1B are greater than five-fold longer than the lifetime of the background. This suggests that the fluorescence lifetime approach offers additional contrast (and potentially better sensitivity) for detecting calcification in the retina.

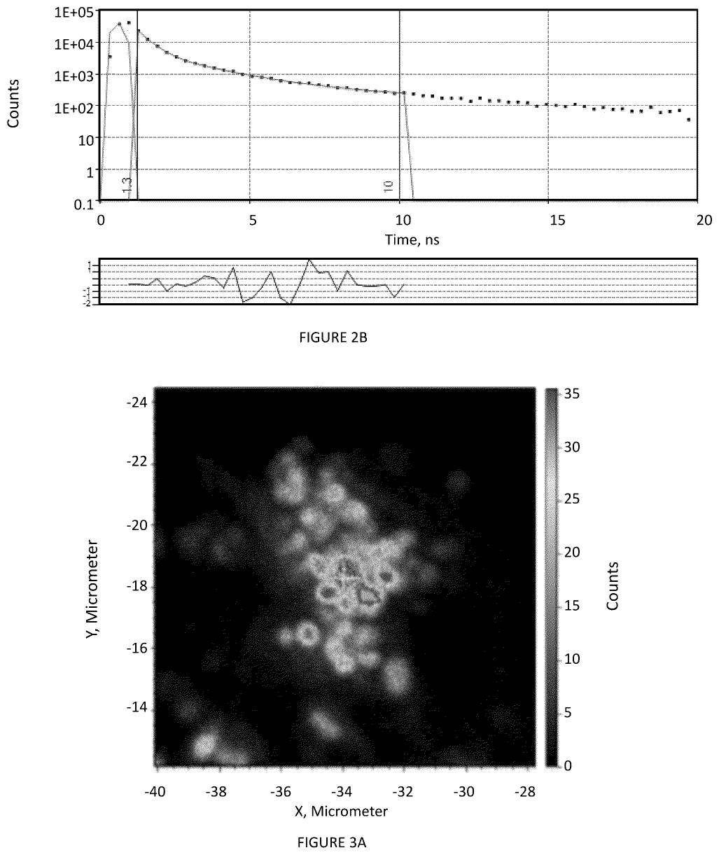

Retinas were also labeled by transarterial infusion post mortem with doxycycline solution, using a similar protocol but less than 250 mg antibiotic/L in the buffer. After perfusion with doxycycline, removal of the neurosensory retina and RPE, and flatmounting, robust staining was observed with two-photon excitation and fluorescence lifetime imaging (FIGS. 2A and 2B).

FIG. 2A depicts a fluorescence intensity micrograph of several small drusen in the retina of a 79-year-old white male donor (cause of death: chronic myelocytic leukaemia) stained with Doxycycline by infusion and imaged with two-photon excitation at 780 nm. FIG. 2B depicts the best two-component fit to the decay in the pink highlighted region of interest centered at X=−34 μm, Y=−18 μm, yielding τ₁=0.430 nsec, α₁=0.88, τ₂=2.111 nsec, α₂=0.12, χ2=0.88. The spots in the small drusen were reminiscent of the micron-sized HAP spherules the current inventors had previously observed in drusen [Thompson, 2015]. Accordingly, it was decided to image them further with FLIM at higher magnification.

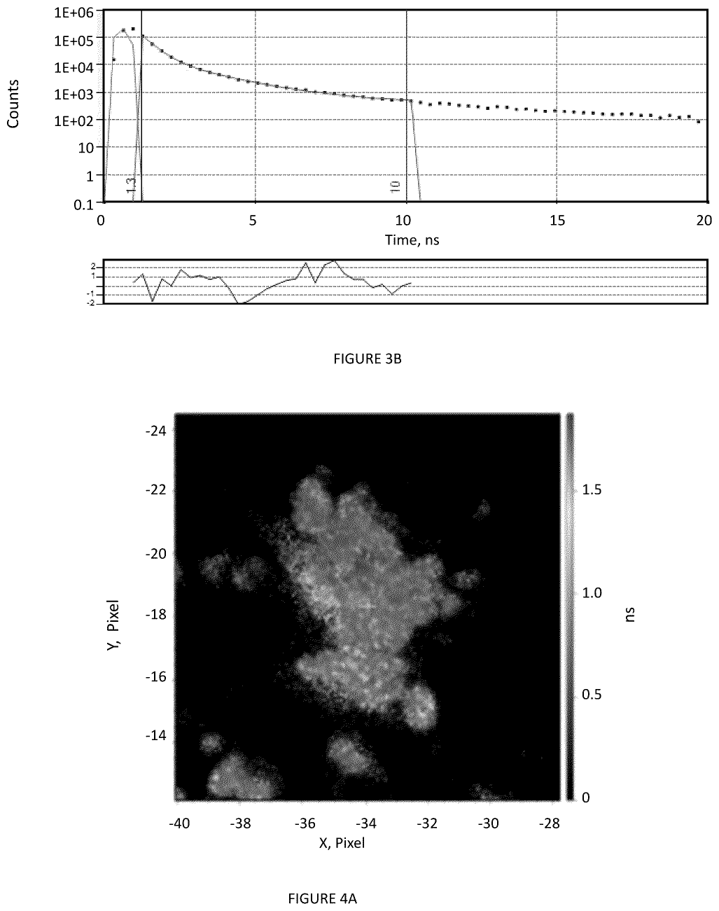

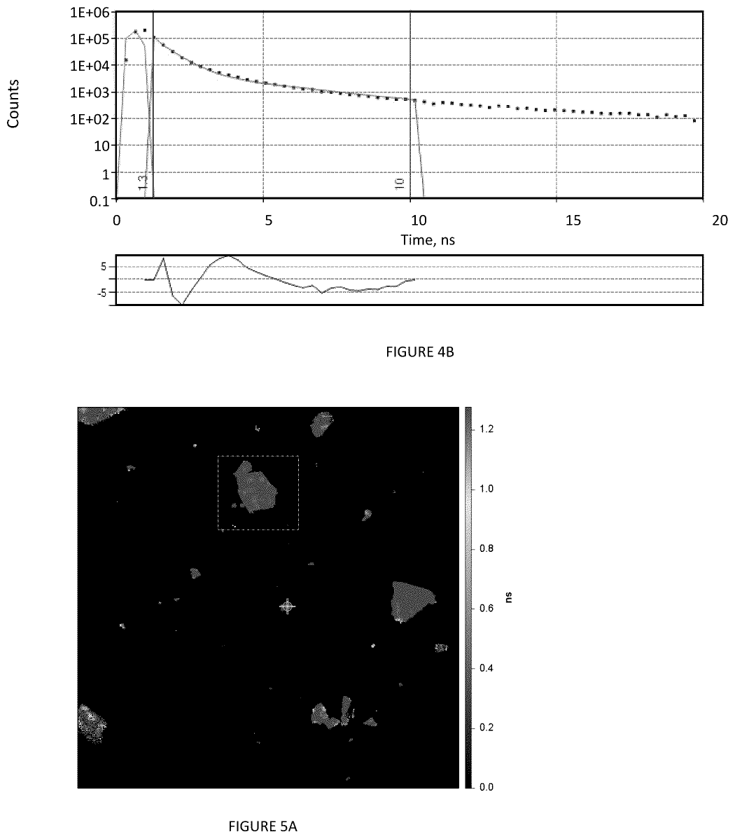

The images in FIGS. 3A and 4A depict a close-up with a 60× objective of the prominent druse near the center of FIG. 2A. The small round blobs clustered in the center of the image are reminiscent in shape and size of the calcium phosphate spherules previously observed elsewhere. Notably, they are distinctly smaller than, and not polygonal, like RPE cells. The time-resolved fluorescence decay of the pink region of interest highlighted in FIG. 3A at X=−33 μm, Y=−17 μm was fit to two components, and the data compared to the best fit in FIG. 3B: the derived values were τ₁=0.417 nsec, α₁=0.95, τ₂=1.737 nsec, α₂=0.05, χ2=2.06. It was further determined if the fit would be significantly worse if the value of the long component were fixed at 3.7 nsec, the lifetime value previously determined for doxycycline bound to hydroxyapatite. The returned values from this fit were τ₁=0.562 nsec, α₁=0.97, τ₂=3.7 (Fixed) nsec, α₂=0.03, χ2=29. As is apparent from the higher χ2 and the larger and systematically varying residuals in FIG. 4B, the fit is worse. In this case it is not clear why the quality of the fit using the known lifetime of doxycycline-stained HAP is not comparable. When the entire image is fit to a single exponential, the image in FIG. 4A is obtained, where the small regions in the druse exhibit a range of fluorescence intensity that is remarkably uniform in their apparent fluorescence lifetime properties. However, with doxycycline staining as well, it is evident that the fluorescence intensities and lifetimes of the stained drusen are several fold greater than the background fluorescence emission, suggesting they will be potentially useful for early imaging of drusen.

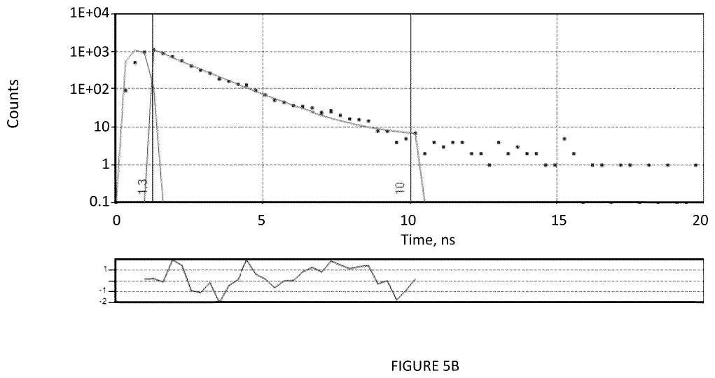

For comparison, FIG. 5A depicts a fluorescence intensity micrograph of synthetic HAP stained with Cl-tetracycline and imaged with two-photon excitation at 780 nm. FIG. 5B depicts the best two-component fit to the decay in the highlighted region of interest, yielding a mean lifetime of=1.31 nsec, χ2=1.4.

Notably, in FIG. 1 it can be seen that a significant portion of the druse (perhaps in addition to the mineral) exhibited a fluorescence lifetime comparable to that of HAP stained with Cl-Tet, the lifetime (or long lifetime component) of doxycycline-stained drusen did not exhibit a significant long lifetime component of significant value. Without being bound by theory, it is assumed that this may be because the bulk of the doxycycline (like at least a portion of the Cl-Tet) adheres to lipid and protein species in the druse as well as HAP, and exhibits a less prolonged lifetime because of this.

Advantageously, the feasibility and safety of two-photon excitation of fluorescence has been demonstrated in the living retinas of animal models and, more recently, in living humans [Feeks, 2017; Palczewska, 2020]. One manufacturer has shown the adaptation of a short-pulse laser to a commercial scanning laser ophthalmoscope [Kamali, 2019] for multiphoton excitation; the principal change required (in addition to the laser and filters) was the insertion of optics to correct for the effects of wavelength dispersion on the pulse duration.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

Denk, W.; Strickler, J. H.; Webb, W. W., Two-photon laser scanning fluorescence microscopy. Science 1990, 248, 73-76.
Feeks, J. A.; Hunter, J. J., Adaptive optics two-photon excited fluorescence lifetime imaging ophthalmoscopy of exogenous fluorophores in mice. Biomed. Opt. Express 2017, 8, (5), 2483-2495.
Göppert-Mayer, M., Über Elementarakte mit zwei Quantensprüngen. Annalen der Physik 1931, 401, (3), 273-294.
Hamer, F. M., The Cyanine Dyes and Related Compounds, Volume 18. Wiley: 1964; Vol. 18.
Hegde, et al., ijms-2176421, in press.
Kamali, T.; Farrell, S. R. M.; Baldridge, W. H.; Fischer, J.; Chauhan, B. C., Two-Photon Scanning Laser Ophthalmoscope. In High Resolution Imaging in Microscopy and Ophthalmology: New Frontiers in Biomedical Optics, Bille, J. F., Ed. Springer Copyright 2019, The Author(s). Cham (CH), 2019; pp 195-211.
Lakowicz, J. R.; Thompson, R. B.; Cherek, H., Phase fluorometric studies of spectral relaxation at the lipid-water interface of phospholipid vesicles. Biochimica et Biophysica Acta (BBA)-Biomembranes 1983, 734, (2), 295-308.
Palczewska, G.; Boguslawski, J.; Stremplewski, P.; Kornaszewski, L.; Zhang, J.; Dong, Z.; Liang, X. X.; Gratton, E.; Vogel, A.; Wojtkowski, M.; Palczewski, K., Noninvasive two-photon optical biopsy of retinal fluorophores. Proc Natl Acad Sci USA 2020, 117, (36), 22532-22543.
Redford, G. I.; Clegg, R. M., Polar plot representation for frequency-domain analysis of fluorescence lifetimes. Journal of Fluorescence 2005, 15, (5), 805-815.
Sarks, et al. (1999) The British Journal of Ophthalmology, 83, pp. 358-368.
Szmacinski, H.; Lakowicz, J. R.; Johnson, M. L., Fluorescence lifetime imaging microscopy: homodyne technique using high-speed gated image intensifier. In Numerical Computer Methods, Johnson, M. L.; Brand, L., Eds. Academic Press: New York, 1994; Vol. 240, pp 723-748.
Szmacinski, H.; Hegde, K.; Zeng, H. H.; Eslami, K.; Puche, A. C.; Lengyel, I.; Thompson, R. B., Imaging hydroxyapatite in sub-retinal pigment epithelial deposits by fluorescence lifetime imaging microscopy with tetracycline staining. J Biomed Opt 2020, 25, (4), 1-11.
Tan, A. C. S.; Pilgrim, M. G.; Fearn, S.; Bertazzo, S.; Tsolaki, E.; Morrell, A. P.; Li, M.; Messinger, J. D.; Dolz-Marco, R.; Lei, J.; Nittala, M. G.; Sadda, S. R.; Lengyel, I.; Freund, K. B.; Curcio, C. A., Calcified nodules in retinal drusen are associated with disease progression in age-related macular degeneration. Science Translational Medicine 2018, 10, (466), eaat4544.
Thompson, R. B.; Reffatto, V.; Bundy, J. G.; Kortvely, E.; Flinn, J. M.; Lanzirotti, A.; Jones, E. A.; McPhail, D. S.; Fearn, S.; Boldt, K.; Ueffing, M.; Ratu, S. G. S.; Pauleikhoff, L.; Bird, A. C.; Lengyel, I., Identification of hydroxyapatite spherules provides new insight into subretinal pigment epithelial deposit formation in the aging eye. Proceedings of the National Academy of Sciences 2015, 112, (5), 1565-1570.
Zinkernagel, M.; Dysli, C., Fluorescence Lifetime Imaging Ophthalmoscopy. Springer: 2019; p 188.

That which is claimed is:

1. A method for identifying or labeling of HAP deposits in tissue in a subject, the method comprising:
administering a hydroxyapatite-selective fluorescent dye to the subject in an amount sufficient for binding to any hydroxyapatite (HAP) deposits or spherules present in tissue to form a HAP/fluorescent dye complex;
exciting the HAP/fluorescent dye complex with electromagnetic radiation (EMR), wherein the EMR has a wavelength in a range from about 700 nm to about 1 mm; and
monitoring and/or measuring the lifetime of a fluorescent signal of the HAP/fluorescent dye complex with a fluorescence lifetime imaging device, wherein the HAP/fluorescent dye complex exhibits a longer fluorescence lifetime signal than that of background fluorescence of the tissue.

2. The method of claim 1, wherein the EMR wavelength is in a range from about 700 nm to about 2500 nm.

3. The method of claim 1, wherein multiphoton excitation is used to irradiate the HAP/fluorescent dye complex.

4. The method of claim 1, wherein two-photon excitation (TPE) is used to irradiate the HAP/fluorescent dye complex.

5. The method of claim 1, wherein the HAP-selective fluorescent dye comprises a species selected from the group consisting of tetracycline chlortetracycline, demeclocycline, doxycycline, methacycline, oxytetracycline, anhydrochlortetracycline, anhydrotetracycline, cetocycline and chelocardin.

6. The method of claim 1, wherein the HAP-selective fluorescent dye comprises chlortetracycline or doxycycline.

7. The method of claim 1, wherein the administration of the HAP-selective fluorescent dye is selected from the group consisting of topically, orally, intravenously, intravitreally, and intraocularly.

8. The method of claim 1, wherein the HAP-selective fluorescent dye comprises chlortetracycline and the presence of a fluorescence lifetime signal of from about 1.4 to about 1.9 nsec indicates the presence of HAP deposits.

9. The method of claim 1, wherein the HAP-selective fluorescent dye comprises doxycycline and the presence of a fluorescence lifetime signal of from about 2.0 to about 4.2 nsec indicates the presence of HAP deposits.

10. The method of claim 1, wherein the fluorescence lifetime imaging device uses fluorescence lifetime imaging microscopy (FLIM) or fluorescence lifetime imaging ophthalmoscopy (FLIO).

11. The method of claim 1, wherein pulsed excitation sources that exhibit pulse durations in the picosecond to femtosecond range are used to excite the HAP/fluorescent dye complex.

12. The method of claim 1, wherein a pharmaceutical composition comprising the HAP-selective fluorescent dye is administered, and the imaging with the fluorescence lifetime imaging device occurs about 8 hours to about 72 hours subsequent to said administration.

13. The method of claim 1, wherein the lifetime of the fluorescent signal is measured by time-domain or by frequency-domain methods.

14. The method of claim 1, further comprising scanning the tissue of the subject with a fluorescence lifetime imaging device.

15. The method of claim 1, further comprising obtaining a profile of HAP deposits in the subject's tissue, wherein the HAP deposits bound to the fluorescent dye exhibit a longer lifetime compared to background tissue.

16. The method of claim 15, further comprising using the obtained profile to diagnose or predict age-related macular degeneration and/or Alzheimer's disease in the subject.

17. The method of claim 1, wherein the method is used in diagnosing or predicting the likelihood of having or developing age-related macular degeneration and/or Alzheimer's disease in the subject.

18. The method of claim 1, wherein the tissue is retina tissue.
